(19) United States
(12) Patent Application Publication
(10) Pub. No.: US 2002/0072679 A1
(43) Pub. Date: Jun. 13, 2002

(54) Intra-aortic balloon catheter having a fiberoptic sensor

(76) Inventors: Robert B. Schock, Sparta, NJ (US); Jonathan Williams, Montville, NJ (US); Daniel A. Walters, Rockaway Twp, NJ (US)

Correspondence Address: Abraham Ronai, Datascope Corp., 14 Philips Parkway, Montvale, NJ, 07645, US

(21) Appl. No.: 09/734,755

(22) Filed: Dec. 12, 2000

(51) Int. Cl. A61B 5/02

(52) U.S. Cl. 600/486; 600/480; 600/561

(57) ABSTRACT

An intra-aortic balloon catheter having a fiberoptic pressure sensor embedded in the tip.

### BACKGROUND OF THE INVENTION

1. Field of the Invention

The invention relates to a catheter having enhanced pressure sensing capabilities. More particularly, the invention relates to a balloon catheter having a fiberoptic sensor connected to the catheter.

2. Description of the Prior Art

A key function of many catheters is that of continuously monitoring blood pressure. In many cases, this monitoring must be performed with accurate measurement of high frequency components. For example, reliable detection of the dicrotic notch of the aortic blood pressure waveform typically requires a pressure signal having a bandwidth of 15 Hz or better. Detection of the dicrotic notch is generally used for the inflation/deflation timing of an intra-aortic balloon (“IAB”) catheter.

Conventional invasive pressure monitoring is performed with low-cost fluid-filled transducers. A typical disposable monitoring kit, inclusive of all tubing, a continuous flush device, and a pre-calibrated transducer is very affordable. Unfortunately, these systems have several drawbacks. One major drawback is that bubbles or clots in the monitoring lines can reduce the frequency response of the system to a level below 15 Hz, creating an “overdamped” condition. In other cases, the characteristics of the catheter and tubing can result in “ringing”, which is associated with an underdamped condition. Furthermore, fluid-filled catheters can suffer from “catheter whip” (motion artifact), which is manifested as one or more high frequency deflections in the pressure signal. These problems can degrade the usefulness of the signal in applications such as intra-aortic balloon pumping (IABP). In particular, it is difficult, if not impossible, to automatically provide optimal timing of IABP using a pressure signal with a frequency response below 15 Hz, or using signals with ringing or whip artifacts that mimic the physiologic dicrotic notch.

Another means for monitoring blood pressure is to use a micromanometer, such as marketed by companies such as Millar, Endosonics, and Radi. See U.S. Pat. Nos. 5,431,628 and 5,902,248, herein incorporated by reference. These devices can have excellent frequency responses, with system bandwidths greater that 200 Hz. They are not subject to the negative effects of bubbles and catheter whip, and retain good performance even in the presence of small blood clots. Unfortunately, they are very expensive, prone to signal drift, and can suffer from electrical interference. A common source of electrical interference in the setting of IABP therapy is the use of electrosurgery. In this situation, it is desirable to maintain a reliable pressure signal with which to trigger the balloon, as the ECG signal which normally triggers IABP operation becomes completely unreliable. Conventional fluid-filled transducer systems are relatively immune from this type of interference. Attempts have been made to use micromanometers for IABP timing, see U.S. Pat. Nos. 3,585,983 and 4,733,652, herein incorporated by reference. These attempts proved to be unreliable, as the device may be damaged during insertion and is also prone to signal drift. To address the drift issue, U.S. Pat. No. 5,158,529, herein incorporated by reference, discloses a method for rezeroing the micromanometer by using the pressure from a partially filled balloon as it rests in the aorta. However, this method requires momentary interruption of IABP, which may be harmful to the critically ill patient.

While standard IAB catheters incorporating a fluid-filled transducer pressure measurement system or IAB catheters incorporating micromanometers may be suitable for the particular purpose employed, or for general use, they would not be as suitable for the purposes of the present invention as disclosed hereafter.

### SUMMARY OF THE INVENTION

Accordingly, there is a need for a reliable and affordable pressure monitoring approach that has high bandwidth pressure sensing, low signal drift, and freedom from electrosurgical interference. There is also a need to incorporate this technology into intra-aortic balloon catheters having small cross sectional profiles.

The invention is an IAB catheter system having enhanced blood pressure sensing capability. The IAB catheter has a fiberoptic sensor built into the tip of the IAB or connected to another part of the catheter.

Fiberoptic sensors have the advantage of being immune to electrical interference, and have the potential to be lower in cost than electronic micromanometers.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is a longitudinal cross sectional view of a distal end of a coaxial IAB catheter having a fiberoptic sensor in the tip.

FIG. 2A is a perspective view of distal end of inner tube 58, shown independent of catheter 10, with pressure sensing line 24 connected to an outer surface of inner tube 58.

FIG. 2B is a perspective view of a distal end of inner tube 58, shown independent of catheter 10, with pressure sensing line sandwiched between an outer surface of inner tube 58 and an outer layer.

FIG. 2C is a perspective view of a distal end of inner tube 58, shown independent of catheter 10, with pressure sensing line 24 embedded in the wall of inner tube 58.

FIG. 3 is a longitudinal cross sectional view of a distal end of a co-lumen IAB catheter having a fiberoptic sensor in the tip.

FIG. 3A is a transverse cross sectional view of the co-lumen IAB catheter in FIG. 3 taken along lines 3A-3A.

FIG. 4A is a longitudinal cross sectional view of the tip having an embedded fiberoptic sensor in a first configuration.

FIG. 4B is a longitudinal cross sectional view of the tip having an embedded fiberoptic sensor in a second configuration.

FIG. 4C is a longitudinal cross sectional view of the tip having an embedded fiberoptic sensor in a third configuration.

FIG. 4D a longitudinal cross sectional view of the tip having an embedded fiberoptic sensor in a fourth configuration.

### DETAILED DESCRIPTION OF THE PREFERRED EMBODIMENTS

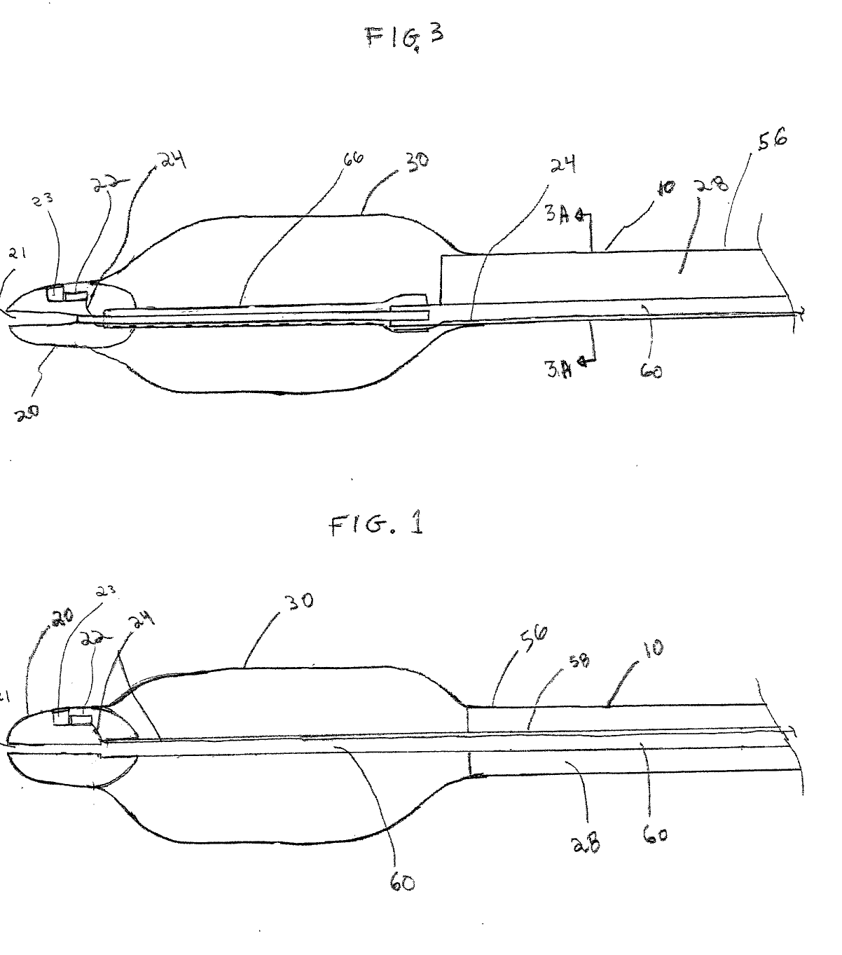

FIG. 1 illustrates a longitudinal cross section of a distal portion of a dual lumen intra-aortic balloon (“IAB”) catheter 10 comprising an outer tube 56, an inner tube 58, a tip 20, and a balloon membrane 30 connected on one end to the outer tube 56 and on the opposite end to the tip 20. Tip 20 defines a tip lumen 21. The inner tube 58 is disposed within the outer tube 56 and is connected to the tip 20 at its distal end. The inner tube 58 defines an inner lumen 60 and the outer tube 56 defines an outer lumen 28. Outer lumen 28 is used for shuttling helium or another appropriate working gas or fluid for inflation and deflation of the balloon membrane 30. The outer tube 56 may be coil or braid reinforced and made from polyurethane or polyimide. Inner tube 58 may be made from polyimide or an alloy with shape memory and superelastic properties commonly referred to as Ni-Ti, NITINOL™, and other industry names. Inner tube 58 may be connected to an inner surface of the outer tube 56 at one or more points to enhance pushability, stability, pumping speed, and pressure fidelity. Furthermore, inner tube 58 may be made from two or more tubes joined end-to-end, as disclosed in U.S. Pat. No. 6,024,693, herein incorporated by reference in its entirety. Fiber optic sensor 22 is embedded in or attached to the tip 20 and preferably has a diameter less than or equal to 0.03 inches. A forward looking end of the fiberoptic sensor 22 faces chamber 23. Pressure sensing line 24 may be embedded in inner tube 58 and connects fiber optic sensor 22 to an optoelectronic interface (not shown) via an industry-standard “ST” connector (not shown). Pressure sensing line 24 preferably is made of glass, but may be made of plastic or another material, and preferably has a diameter less than or equal to 0.006 inches. Note that the fiberoptic sensor 22 may be positioned in alternate locations along IAB catheter 10 as well as on a distal tip of an independent catheter that can be disposed within the inner lumen 58.

FIGS. 2A-2C illustrate perspective views of a distal end of inner tube 58 with pressure sensing line 24 connected to inner tube 58 in various configurations. In FIG. 2A, pressure sensing line 24, is connected to an outer surface of inner tube 58. In FIG. 2B, pressure sensing line 24 is disposed between inner tube 58 and a thin walled tube 64, which preferably is heat shrinkable. Heat shrinkable tubing with a wall thickness of less than or equal to 0.001 inches is available from Advanced Polymers, Inc., Salem, N.H. In FIG. 2C, pressure sensing line 24 is embedded in the wall of inner tube 58. Note that although pressure sensing line 24 is shown running along a longitudinal axis of inner tube 58 it may also be wound helically.

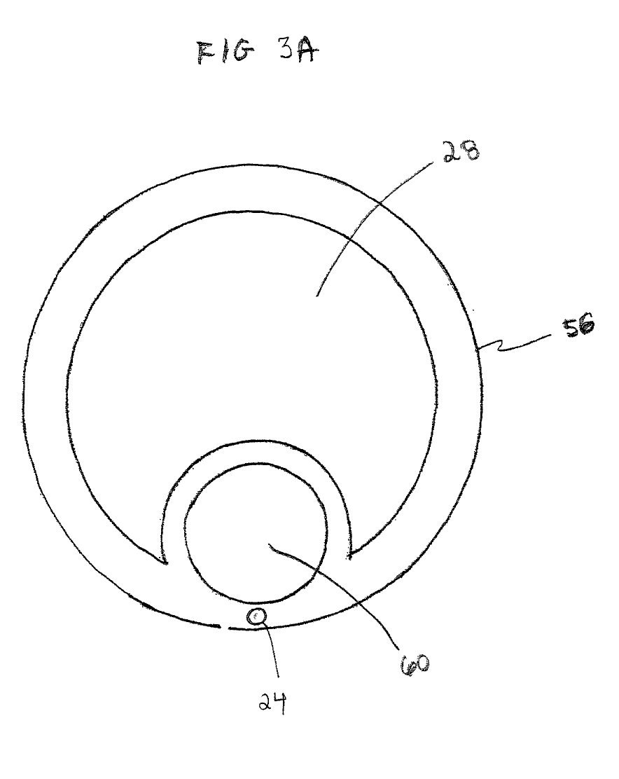

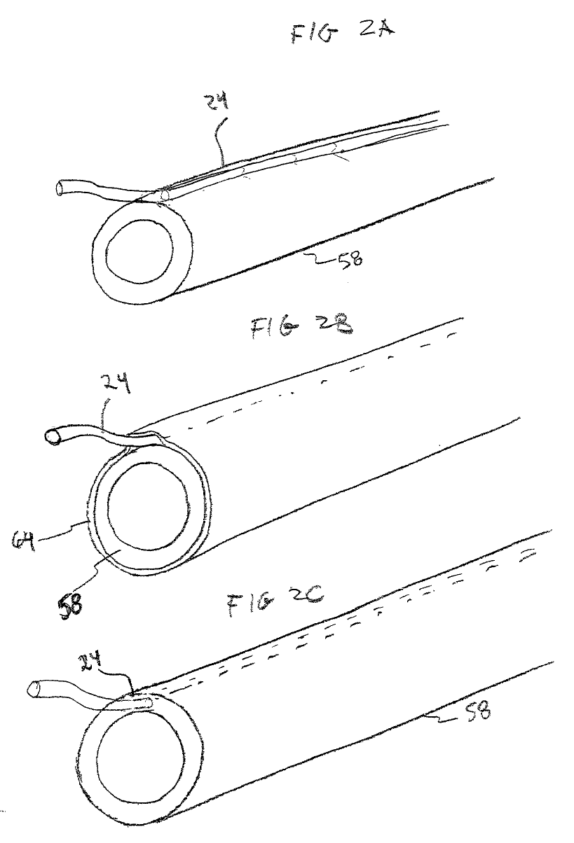

FIG. 3 illustrates a distal portion of another embodiment of the IAB catheter 10, comprising a balloon membrane 30, a tip 20, a co-lumen tube 56, an inner lumen extension tube 66, and a fiberoptic sensor 22. Detailed structure of a co-lumen IAB is disclosed in U.S. Pat. No. 6,024,693 and U.S. Pat. application Ser. No. 09/412,718, filed on Oct. 5, 1999, both herein incorporated by reference. Tip 20 is connected to a distal end of the balloon membrane 30 and to a distal end of the inner lumen extension tube 66. Tip 20 defines a tip lumen 21. A distal end of the co-lumen tube 56 is connected to a proximal end of the balloon membrane 30 and to a proximal end of the inner lumen extension tube 66. The co-lumen tube 56 may be coil or braid reinforced and made from polyurethane or polyimide. The preferred material for inner lumen extension tube 66 is an alloy with shape memory and superelastic properties commonly referred to as Ni-Ti, NITINOL™, and other industry names. Inner lumen extension tube 66 may also be made from polyimide. The fiberoptic sensor 22 is attached to tip 20 and pressure sensing line 24 which communicates signals generated by the fiberoptic sensor 22 to an optoelectronic interface (not shown). The pressure sensing line 24 may be connected to the inner lumen extension tube 66 and co-lumen tube 56 in any of the ways pressure sensing line 24 is connected to the inner tube 58, illustrated in FIGS. 2A-2C. Furthermore, the pressure sensing line may be embedded in the outer tube 56 as illustrated in FIG. 3A.

FIG. 3A illustrates a transverse cross section of outer tube 56, taken along line 3A-3A illustrated in FIG. 3, with pressure sensing line 24 embedded in the wall. Outer tube 56 defines two distinct lumens, inner lumen 60 and outer lumen 28. Outer lumen 28 is used for shuttling helium or another appropriate fluid or gas for inflation and deflation of balloon membrane 30. Note that pressure sensing line 24 may be embedded at a different location in outer tube 56, may be connected to a surface of outer tube 56, or may freely reside in inner lumen 60. Note also that the fiberoptic sensor 22 may be positioned in alternate locations along IAB catheter 10 as well as on a distal tip of an independent catheter that can be disposed within the inner lumen 58.

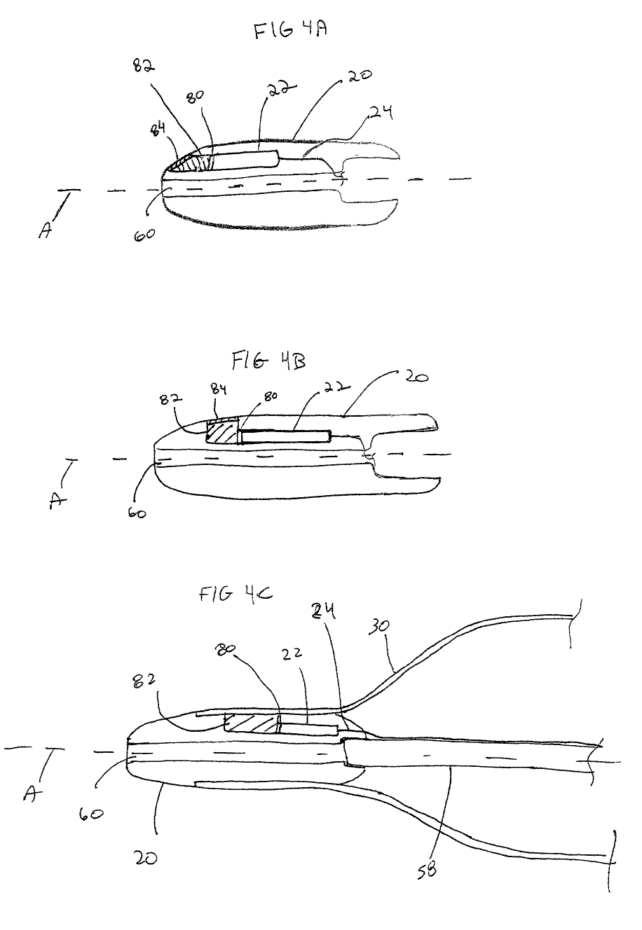

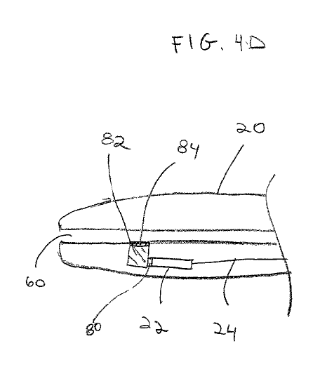

FIGS. 4A-4C illustrate alternate configurations for tip 20 incorporating a forward looking fiber optic sensor 22. In FIG. 4A and FIG. 4B longitudinal cross sections of tip 20 are shown independent of the rest of catheter 10 for clarity. In FIG. 4A fiber optic sensor is embedded in tip 20 parallel to the longitudinal axis of tip 20 labeled A. Fiber optic sensor 22 has an approximate outer diameter of 0.0025″ and has a pressure sensing surface or diaphragm 80 on a distal end which is exposed to protective pocket 82. Diaphragm 80 may be made from silicone approximately 6 microns thick and is protected from physical damage and blood contact by protective pocket 82, which may contain a gel, fluid, gas, elastomer, or any other flexible protective material. Diaphragm 80 may project into the pocket or may be flush with the protective pocket 82 wall. Membrane 84 prevents leakage of gel or protective pocket 82. Pressure sensing line 24 comprises a fiberoptic fiber having an outer diameter of approximately 0.006″. In FIG. 4B (and also FIGS. 1 and 3) the gel or protective pocket 82 is shifted proximally. In FIG. 4C a distal portion of balloon membrane 30 is used to seal protective pocket 82. In FIG. 4D protective pocket 82 opens into inner lumen 60. Membrane 84 prevents leakage of gel or protective pocket 82. In this location fiberoptic sensor 22 is exposed to arterial pressure via tip lumen 21 and is less likely to be damaged upon insertion and placement of IAB catheter 10. Note that although forward looking fiber optic pressure sensors are illustrated, use of side looking sensors is anticipated as well.

In addition to sensing blood pressure, the fiberoptic sensor 22 can be used to measure other important physiologic variables, such as, but not limited to temperature (as in the Luna Innovations sensor noted below), pCO2, pO2, pH, oxygen saturation, lactate, CPK, anesthetic agent concentration, various biological compounds, electrolytes, and nitric oxide. Blood pressure, as well as at least some of these variables, could be measured using either extrinsic or intrinsic fiberoptic sensors. Extrinsic sensors use the fiberoptic fiber to communicate information from a separate structure which is attached to the fiber. Intrinsic sensors sense information as a result of physical changes of the fiber itself. Intrinsic sensors have a particular advantage of being extremely small, rugged, and low in cost.

As indicated above the pressure sensing line 24 terminates in an optoelectronic interface which provides a light source, light detector, optical components (such as a lens, splitter, and interferometer), power supply, and signal processing means. This optoelectronic interface is known in the art and can be connected to or integrated with another electronic device, such as a monitor or intra-aortic balloon pump. Light originating from the interface travels down the fiberoptic fiber, i.e. the pressure sensing line 24, by the well-known principal of total internal reflection to the sensing element. The sensing element incorporates pressure responsive means which modifies the character of the light as a function of pressure. The modified light is in turn reflected back through the fiber to the interface. The optical components, light detector, and signal processing elements of the interface work together to produce an electrical output that characterizes the pressure at the sensor. This output is translated into a displayed pressure waveform or digital pressure display by the IABP or monitor.

Fiberoptic pressure monitoring sensors and optoelectronic interfaces suitable for this application are manufactured by several companies, including FISO Technologies Inc. (Quebec, Canada) and Luna Innovations (Blacksburg, Va.). FISO utilizes a white light interferometric approach, as described in U.S. Pat. Nos. 5,202,939 and 5,392,117, herein incorporated by reference in their entirety. Luna Innovations uses a laser light source in an approach similar to that described in U.S. Pat. No. 5,301,001, herein incorporated by reference in its entirety.

There are a variety of possible configurations for the fiberoptic sensor itself. One type would employ a deformable diaphragm separated from the end of one or more fiberoptic fibers by a chamber. The chamber may be filled with air and vented to atmosphere to eliminate temperature-induced pressure changes within the chamber (these would be associated with signal drift). A disadvantage of this design in an IAB catheter is that an air-venting tube is required which occupies a portion of the cross-sectional area of the catheter. This tube reduces the potential to produce the catheter in a small size with acceptable performance.

A more desirable approach to a fiberoptic pressure sensor for a small catheter is to utilize a design without a vent tube (as illustrated in FIGS. 1, 3, and 4A-4D). Such a sensor can be produced by positioning the sensor diaphragm over a chamber with a vacuum (as currently practiced by FISO). With this approach, a sensor can be constructed which has little temperature-induced drift, assuming that the materials which are used to build the diaphragm and chamber have small coefficients of thermal expansion (ceramics, glass, and silicon are good choices). This approach makes the device an absolute pressure sensor, which requires a separate barometric pressure sensor to convert absolute pressures to gauge pressures. In IABP applications, such a barometric pressure sensor could be built into the pump. U.S. Pat. No. 5,158,529, herein incorporated by reference in its entirety, teaches another possible means for assuring the accuracy of gauge pressure values as indicated by a sensor in the IAB catheter; this approach compares the blood pressure values indicated by the catheter sensor with pressure values in the gas within a partially-filled catheter and pump, and adjusts the sensor-indicated pressure to agree with the gas pressure.

Another approach to constructing a non-vented pressure sensor is to trap a gas in the chamber beneath the diaphragm and to monitor its temperature (as currently practiced by Luna Innovations). In this case, knowledge of the gas temperature within the chamber allows for correction of temperature-induced pressure changes. Ideally, if a gas is to occupy the chamber, it is dry and inert. It is possible that appropriate selection of the bias gas would improve linearization of the sensor or change its dynamic range.

As many apparently widely different embodiments of the present invention can be made without departing from the spirit and scope thereof, it is to be understood that the invention is not limited to the specific embodiments thereof except as defined in the appended claims.

What is claimed is:

1. A balloon catheter comprising a balloon membrane, a tip, a fiberoptic sensor connected to said tip, a fiberoptic fiber, an outer tube, and an inner tube disposed within an outer surface of said outer tube, said inner tube extending beyond a distal end of the outer tube, a distal end of the balloon membrane being connected to the tip and to a distal end of the inner tube, said fiberoptic fiber being connected on a distal end to the fiberoptic sensor and proximal to the fiberoptic sensor being at least partially connected along its length to the inner tube.

2. The balloon catheter as claimed in claim 1 wherein the fiberoptic fiber is sandwiched between the inner tube and a thin walled tube disposed over the inner tube.

3. The balloon catheter as claimed in claim 2 wherein the thin walled tube is heat shrunk over the fiberoptic fiber and inner tube.

4. The balloon catheter as claimed in claim 1 wherein the fiberoptic fiber is embedded in the inner tube.

5. The balloon catheter as claimed in claim 1 wherein the fiberoptic fiber is adhered to an outer surface of the inner tube.

6. The balloon catheter as claimed in claim 1 wherein the inner tube is connected to the outer tube, an inner surface of the outer tube defines an outer lumen, and the fiberoptic fiber has a balloon portion which is disposed with the balloon membrane and an outer tube portion which is disposed within the outer tube, the balloon portion of the fiberoptic fiber is connected to the inner tube, the outer tube portion of the fiberoptic fiber is disposed within the outer lumen.

7. The balloon catheter as claimed in claims 1 or 6 wherein the inner tube is connected to the outer tube and comprises two tubes connected end-to-end.

8. The balloon catheter as claimed in claim 1 wherein the tip comprises an inner surface, an outer surface, and a pocket, said inner surface defining a tip lumen extending from a proximal end of the tip to a distal end of the tip, and wherein the fiberoptic sensor has a pressure sensing surface, the fiberoptic sensor is embedded in the tip such that the pressure sensing surface is exposed to said pocket.

9. The balloon catheter as claimed in claim 8 wherein the pocket extends from the outer surface of the tip to a point between the inner surface of the tip and the outer surface of the tip.

10. The balloon catheter as claimed in claim 8 wherein the outer surface of the tip comprises a distal sloping portion and wherein the pocket extends from said distal sloping portion to a point between said distal sloping portion and said proximal end of said tip.

11. The balloon catheter as claimed in claim 8 wherein the pocket extends from a point between the inner surface of the tip and the outer surface of the tip to the inner surface of the tip such that it communicates with the inner lumen.

12. The balloon catheter as claimed in claims 8, 9, 10, or 11 wherein the pocket is filled with a protective material.

13. The balloon catheter as claimed in claims 8, 9, 10, or 11 wherein the pocket is filled with a gel.

14. The balloon catheter as claimed in claims 8, 9, 10, 11, or 12 wherein the pocket is sealed by a membrane.

15. The balloon catheter as claimed in claims 8, 9, 10, 11, or 12 wherein the pocket is sealed by the balloon membrane.

16. A balloon catheter system comprising a fiberoptic sensor catheter and a balloon catheter, said balloon catheter comprising a balloon membrane, a tip having a tip lumen, an outer tube, and an inner tube disposed within an outer surface of said outer tube, said inner tube extending beyond a distal end of the outer tube, a distal end of the balloon membrane being connected to the tip and to a distal end of the inner tube, said fiberoptic sensor catheter comprising a tube having a fiberoptic sensor connected to a distal end, said fiberoptic sensor being connected to a distal end of a fiberoptic fiber which is connected to the tube, said fiberoptic sensor catheter fitting within the inner tube and in the tip lumen.

17. The balloon catheter as claimed in claim 16 wherein the inner tube is connected to the outer tube.

18. An intra-aortic balloon catheter comprising a co-lumen tube, a balloon membrane, an inner lumen extension tube, and a tip, said co-lumen tube having an outer lumen inner surface, defining an outer lumen, and an inner lumen inner surface, defining an inner lumen, said inner lumen having a smaller cross sectional area than said outer lumen, a proximal end of the inner lumen extension tube and a proximal end of the balloon membrane are connected to a distal end of the co-lumen tube, the tip, a distal end of the inner lumen extension tube, and a distal end of the balloon membrane are connected, said tip having an outer surface, an inner surface, defining an inner tip lumen, and a pocket, a fiberoptic sensor is embedded in the tip such that a pressure sensing surface of the fiberoptic sensor is exposed to said pocket.

19. The intra-aortic balloon catheter as claimed in claim 18 wherein the pocket is filled with a protective material and wherein the pocket is sealed by a membrane.
